(19) United States
(12) Patent Application Publication
(10) Pub. No.: US 2006/0280820 A1
(43) Pub. Date: Dec. 14, 2006

(54) Use of an extract of Aloysia/Verbena/Lippia triphylla/citriodora for the treatment of chronic and/or inflammatory diseases

(76) Inventors: Karim Balan, Regensburg (DE); Dietrich Paper, Maxhutte-Haidhof (DE)

Correspondence Address: JENKINS, WILSON, TAYLOR & HUNT, P. A., 3100 TOWER BLVD, SUITE 1200, DURHAM, NC, 27707, US

(21) Appl. No.: 11/454,777

(22) Filed: Jun. 16, 2006

Related U.S. Application Data
(63) Continuation of application No. PCT/EP04/14499, filed on Dec. 20, 2004.

(30) Foreign Application Priority Data
Dec. 18, 2003 (DE) 10359384.5

(51) Int. Cl. A61K 36/85

(52) U.S. Cl. 424/774

(57) ABSTRACT

This invention relates to the use of an extract of Aloysia triphylla or a fraction thereof or of a lyophilisate thereof, or of one or more active ingredients of the extract, or of an Aloysia triphylla extract as a matrix protector for inhibiting the angiogenesis of different geneses, for chemoprevention, and for treating chronic diseases such as cancer, rheumatoid arthritis, inflammatory intestinal diseases, psoriasis and neurological diseases in which the pathogenesis is caused by reactive oxygen species. The invention also relates to an extract produced from Aloysia triphylla.

### RELATED APPLICATIONS

This application is a continuation of PCT International Patent Application No. PCT/EP2004/014499, filed Dec. 20, 2004, which claims priority to German Patent Application No. 10359384.5, filed Dec. 18, 2003, the disclosures of each of which are incorporated herein by reference in their entitety.

### TECHNICAL FIELD

The invention relates to the use of an extract of Aloysia triphylla (L'Her.) O. Kuntze/Britt. (syn. Lippia citriodora H. B. K., Lippia triphylla (L'Her.) O. Kuntze) or a fraction of the same or a lyophilisate thereof, respectively, or of one or more active ingredients of the extract or an Aloysia triphylla extract which is adjusted in e.g. flavonoids or anti-oxidative properties (e.g. trolox equivalents) as a matrix protector for inhibiting the angiogenesis of different geneses and for the chemoprevention and treatment of chronic diseases such as e.g. cancer, rheumatoid arthritis, inflammatory intestinal diseases (e.g. Morbus Crohn), psoriasis and neurological diseases having a pathogenesis caused by reactive oxygen species (e.g. Alzheimer). Furthermore, the present invention relates to an extract prepared from Aloysia triphylla.

### BACKGROUND

For Aloysia triphylla, several synonymous designations exist in the literature: Aloysia triphylla (L'Hérit.) Britt./Lippia citriodora H. B. K/Verbena triphylla L'Herit., Lemon Verbena, Herb Louisa, among others.

### BRIEF DESCRIPTION OF THE FIGURES

The following is shown in the Figures:

FIG. 1 shows a flow chart of the preparation of an extract according to the invention;

FIG. 2 shows the inhibition of the degradation of glycosaminoglycanes (e.g. chondroitinsulfate A) by reactive oxygen species (ROS) by the water-soluble Aloysia extract according to the invention. In contrast, the anti-oxidative vitamin C enhances the degradation of glycosaminoglycanes under physiological conditions (pH value 7.2, 37 degrees Celsius);

FIG. 3 shows the impact of a water-soluble Aloysia extract according to the invention on physiological angiogenesis as compared to vitamin C;

FIG. 4 represents the inhibition of pathological angiogenesis caused by chronic inflammation by means of a water-soluble extract from Aloysia according to the invention in comparison to vitamin C;

FIG. 5 shows the inhibition of the irritation at the chorioallantoic membrane caused by reactive oxygen species (ROS).

### DETAILED DESCRIPTION

The inventors have surprisingly found that a novel extract obtained from Aloysia triphylla on the basis of hydrophilic solvents has a wide variety of therapeutic applications which so far have neither been attributed to the plant in its entirety nor to fractions thereof.

Thus, the invention relates to the use of an extract of Aloysia triphylla, preferably of the dry extract, the mother tincture, the fluid extract or a fraction of the same or a lyophilisate thereof, respectively, or of one or more of the active ingredients of the extract as a matrix protector for the inhibition of pathogenic angiogenesis as well as in particular for the treatment and prevention of tumor diseases, rheumatoid arthritis and other chronic diseases such as e.g. Alzheimer, psoriasis, retinopathies and periodontitis of the teeth wherein this, however, is only an exemplary listing and future therapeutic applications are taken into consideration also in other fields where inhibition of angiogenesis plays a role.

The key feature of the extract of Aloysia triphylla according to the invention is that the extract is obtained from the vegetal starting material by one or more hydrophilic solvents and is essentially completely soluble in water.

Among others, the extract shows an excellent anti-inflammatory effect and can therefore find use in various fields, e.g. in medicine, cosmetics, etc.

As already mentioned above, the extract can exist in many forms which are already known e.g. as dry extract, mother tincture, fluid extract, specific extract or the lyophilisates thereof. The term “extract” as used herein also comprises fractions, i.e. active agent-containing subgroups of the extract which were e.g. obtained by further treatment with individual hydrophilic solvents.

The preparation of a specific extract of Aloysia triphylla having a high content of active ingredients is for example achieved following extraction with solvents on the basis of water/alcohol followed by partitioning with organic solvents such as e.g. acetone, chloroform, dichloromethane, ethylacetate etc. and subsequent chromatographic purification e.g. on silica gel or RP18 material.

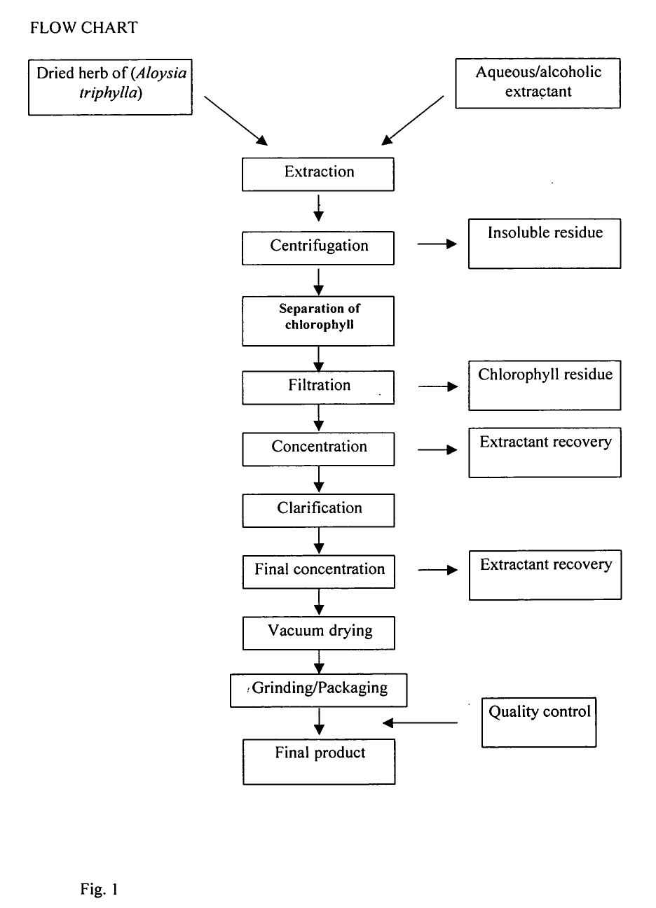

The preparation of a completely water-soluble specific extract from Aloysia triphylla is shown in FIG. 1. The process of preparation is also explained in the Examples.

Fluid extracts and specific extracts (completely water-soluble extracts adjusted to a minimum content in anti-oxidative oligosaccharides) and the preparation thereof are known in the prior art. Those skilled in the art will at any time be able to vary the conditions of the preparation to obtain useful compositions. Relevant protocols can be found in particular in DAB 2004 or EAB 4, 7^{th} edition, supplement. Further relevant technical information is contained in text books of pharmaceutical technology, e.g. in “Pharmazeutische Technologie”, Rudolf Voigt, 9^{th} completely revised edition, or in “Remington's Pharmaceutical Sciences”, Mack Publishing Co., Easton, Pa., 18^{th} edition.

The Aloysia extracts and fractions thereof described herein are extracts obtained by aqueous or aqueous-ethanolic extraction, respectively, from the above ground plant parts of Aloysia triphylla whereafter they are capable of being essentially completely dissolved in water. These preparations are characterized by a particular content of non-volatile, hydrophilic anti-oxidative oligosaccharides. This is a substantial difference as compared to the other Aloysia extracts used in the market so far which are obtained by water vapour distillation and contain volatile, lipophilic essential oils.

The term “anti-oxidative oligosaccharides” as used herein means oligosaccharides which e.g. are di-, tri- to nonasaccharides and are substituted by anti-oxidative groups, e.g. caffeic acid(s), 3,4-dihydrophenyl ethanol, luteoline etc. At this point it shall be pointed out that after comprehensive research work the inventors have found that these anti-oxidative groups alone do not show the effect of the total extract. It is the mixture of the anti-oxidative oligosaccharides present in the extracts according to the invention which develop an optimal effect.

The specific ethanol-water mixtures used, the extraction temperature, the extraction period and the amount of extractant vary between batches of the vegetal starting material and are dependent on the content in anti-oxidative oligosaccharides and the content of undesired interfering substances (such as e.g. chlorophyll, carotenoids and other, in particular lipophilic, components). Thus, the method for obtaining the extracts according to the invention must be adapted, as appropriate, depending on the type of vegetal starting material used which of course will not rise any problems for those skilled in the art of phytopharmaceutics.

Two important criteria to be considered in the method of preparation are the following:

a) no temperatures of more than 90° C. must be used in the extraction method since this leads to hydrolysis of the active agents and therefore to a reduction or even complete elimination of the effect of the extract;

b) the extracts obtained must be essentially soluble in water without any residue.

If in individual cases the question arises whether an extract has the expected (optimal) effects or not, the Aloysia extract can be examined for its effect by means of the HET-CAM assay (described below) and optionally can be adapted (biological standardisation).

The present invention in particular relates to the following aspects and embodiments:

According to a first aspect, the invention relates to an extract of Aloysia triphylla or a fraction thereof wherein the extract is obtained by one or more hydrophilic solvents and is capable of being essentially completely dissolved in water. In the present invention it is most preferably that the extract is soluble in water without any residue which, however, may not be completely achieved in some cases due to the method of extraction chosen. It should be understood that also those extracts still are in the scope of the present invention.

The hydrophilic solvent used for the extraction preferably is water and/or ethanol. The solvent contains water in a range of 100% V/V to 30% V/V while the rest usually is ethanol.

According to a preferred embodiment the extract is a dry extract, fluid extract or a specific extract.

The invention also relates to an extract which can be prepared according to the method of preparation shown in FIG. 1.

The extract according to the invention can also be in a lyophilised form to achieve an as efficient storage and subsequent reconstitution in water (e.g. Aqua ad Injectabilia) as possible.

According to a second aspect, the invention relates to foodstuffs, nutraceuticals or cosmetics containing an extract of Aloysia triphylla or a fraction thereof such as defined above. Some examples for the application of the extract in the field of foodstuffs can be found in Example 4.

According to a third aspect the present invention relates to a pharmaceutical composition containing an extract of Aloysia triphylla or a fraction thereof as defined above and one or more pharmaceutical adjuvants/carriers.

This pharmaceutical composition preferably is intended for administration by injection, systemic and/or topic administration.

According to a fourth aspect, the present invention relates to the use of an extract of Aloysia triphylla or a fraction of the same or a lyophilisate thereof, respectively, or of one or more active ingredients or an Aloysia triphylla extract adjusted for e.g. flavonoids or anti-oxidative properties (e.g. trolox equivalents) for inhibiting pathogenic angiogenesis and for the chemoprevention and treatment of chronic diseases such as e.g. cancer, rheumatoid arthritis, chronic intestinal diseases (e.g. Morbus Crohn), psoriasis and neurological diseases in which the pathogenesis is caused by reactive oxygen species (e.g. Alzheimer).

As mentioned above, the extracts according to the invention are obtained from the whole plant drug (“Herba”), i.e. from all above ground plant parts (stem, leaves, flowers). Therefore, also essential oils can be contained in the compositions according to the invention although they do not represent the major portion of active agents.

Furthermore, the present invention relates to the use of an extract of Aloysia triphylla or a fraction of the same or a lyophilisate thereof or of one or more active ingredients or an Aloysia triphylla extract adjusted in e.g. flavonoids or anti-oxidative properties (e.g. trolox equivalents), respectively, for the protection against degradation of the cartilage in e.g. joints and in the extracellular matrix (matrix protector).

Preferably, Aloysia triphylla finds use in the inhibition of angiogenesis for the treatment and prevention of inflammatory diseases, tumor diseases, rheumatoid arthritis and other chronic diseases such as e.g. Alzheimer, psoriasis, retinopathy and periodontitis of the teeth.

According to the invention, the extract is preferably present as a fluid extract. As mentioned above, the dosage of the fluid extract is between 20 mg and 2 g per day based on the dry substance.

The extracts etc. according to the invention find use as a botanical, in particular as a medicament, nutraceutical, functional food, novel food, cosmetic component (e.g. in sun protection creams, anti-ageing creams und ointments, after shave, hair care compositions etc.) and in foodstuffs having anti-oxidative properties. In other words, the present invention is not directed to the use of the already known and also widely used essential oils.

Furthermore, the invention relates to a lyophilisate prepared from an extract of Aloysia triphylla or a fraction of the same as defined above as well as a botanical, nutraceutical or cosmetic containing an extract of Aloysia triphylla or a fraction thereof.

Finally, the invention relates to a method for the preparation of the extracts according to the invention.

According to the invention, coated tablets, hard gelatine capsules, liquid preparations of the dry substance of the fluid extract are preferably used as forms of presentation as orals, topic forms of use or injectables.

Although the main intended use is in humans, a use in the veterinary field is also possible.

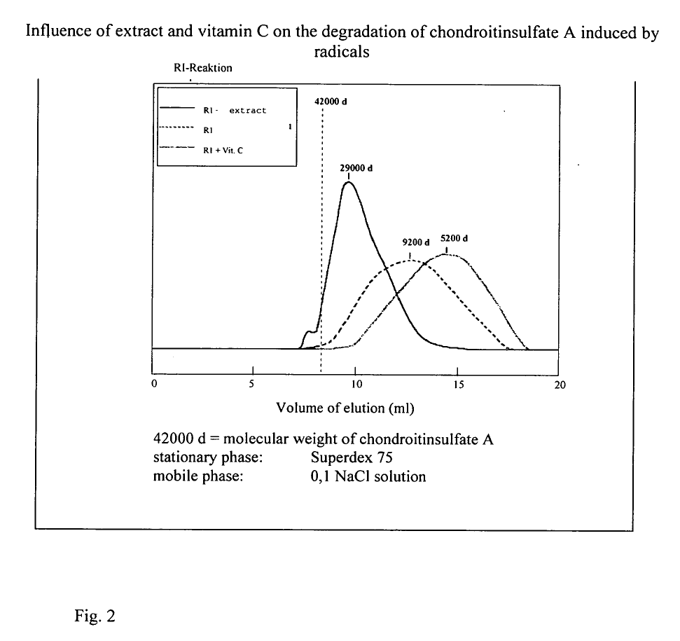

The Aloysia extracts have strong anti-oxidative properties—as shown in the DPPH test. Surprisingly, however, they do not have pro-oxidative properties such as e.g. vitamin C which would lead to degradation of glycosaminoglycanes (e.g. heparin, chondroitinsulfates, heparansulfates). The extracts inhibited the degradation of glycosaminoglycanes caused by free ferrous ions and hydrogen peroxide (see FIG. 2).

Due to this novel effect of the Aloysia extracts they are able to slow or to arrest, respectively, the degradation of e.g. cartilage or the degradation of the extracellular matrix which are essential features in the pathogenesis of a number of chronic diseases. The degradation of glycosaminoglycanes which are components of the extracellular matrix is of high importance for the induction of angiogenesis. Active agents or extracts inhibiting the degradation of the extracellular matrix (matrix protectors) have not been known up to now.

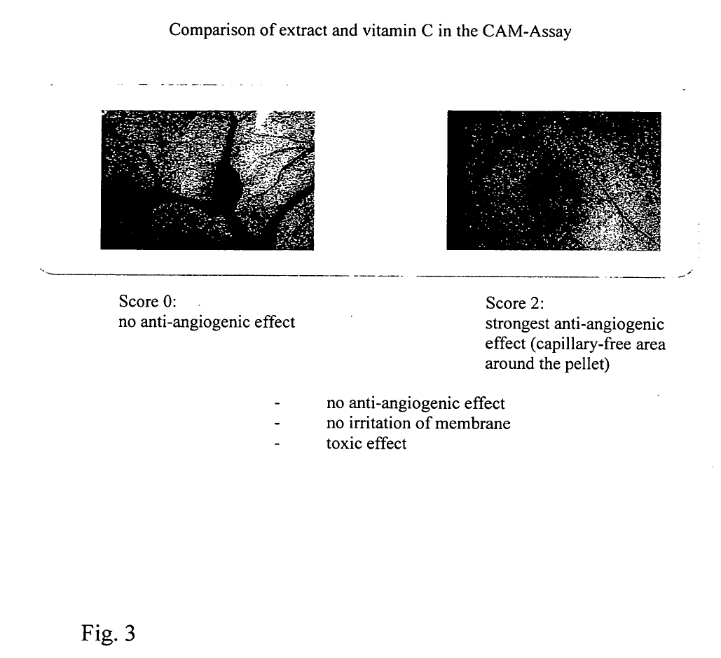

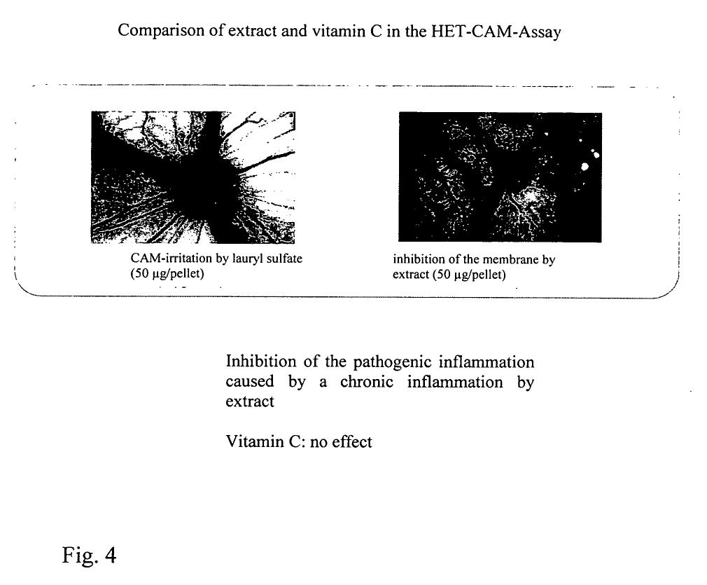

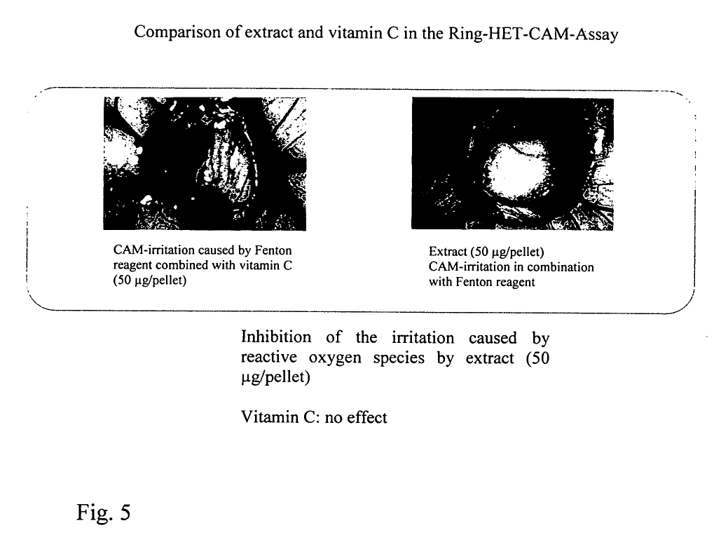

The pharmacological effects of the described Aloysia extracts supposed from the in vitro experiments have been proven in vivo by inhibiting the pathological angiogenesis of different geneses using the chorioallantoic membrane (HET-CAM) of the brooded hen's egg (see FIG. 4). In addition, the irritation caused by reactive oxygen species (ROS) at the chorioallantoic membrane is inhibited (see FIG. 5). Physiological angiogenesis is not inhibited by the Aloysia extracts (see FIG. 3).

An intact extracellular matrix is essential for healthy tissue. Degradation of the components causes e.g. cartilage degeneration or the induction of pathogenic angiogenesis.

Angiogenesis is a physiologically differentiating tissue process in embryonic development, following female menstruation or in wound healing. This differentiation is initiated by capillaries in which the basal lamina is locally destroyed, endothelial cells migrate and proliferate, form a tube as well as a loop with adjacent sites of proliferation. The basal lamina is formed at the newly generated vessels. This process is subject to regulation by antagonising mediators. Among the angiogenesis-stimulating factors are acidic fibroblast growth factor (FGF-1), basic fibroblast growth factor (FGF-2), vascular endothelial growth factor (VEGF), interleukin 1a (IL 1a) among others. Endogenous inhibitors antagonise these stimulating factors and prevent angiogenesis in the healthy individual.

Angiogenesis plays a role in pathogenesis in several diseases. These include above all tumor diseases. Both the growth of a solid tumor and metastases are dependent on angiogenesis in the tumor tissue. Several other examples of diseases in which angiogenesis plays a pathogenic role are already mentioned above.

Angiogenesis inhibitors which are effective and have low side effects still are a therapeutic need today since no angiogenesis inhibitor approved for the use in humans is yet available. Although different angiogenesis inhibitors, e.g. suramin, have already been clinically studied the therapeutic benefit has been doubted due to toxic effects.

With the pharmacological examination of the lyophilisate of the fluid extract of Aloysia triphylla at the CAM of the hen's embryo a strong inhibition of pathogenic angiogenesis was surprisingly obtained. No membrane-irritating or toxic effect of the extract has been observed. No side effects in the use of Aloysia triphylla have been reported to date.

Angiogenesis inhibitors are sought in novel therapeutic strategies for the treatment of rheumatoid arthritis since in the pathogenesis synovial proliferation is accompanied by neovascularisation. Suppression of the proliferation of endothelial cells can be considered as an important therapeutic aim since thereby also a reduction of the pathological-immunological process can be expected.

### EXAMPLES

### Example 1

### Preparation of a Completely Water-Soluble Specific Extract From Aloysia triphylla (see also FIG. 1)

The dried, cut plant material of Aloysia triphylla (above ground components with or without flowers, or leaves or stems, respectively, alone) containing a minimum amount of anti-oxidative oligosaccharides are extracted with water or with water/ethanol mixtures (e.g. water/ethanol=50/50 (V:V)) at 40 degrees Celsius over about 8 hours. The ratio of dried plant material to extractant can vary and is 1:4 to 1:100 parts. The specific ethanol-water mixtures, extraction temperature, extraction period and the amount of extractant used vary between batches and are dependent on the content of anti-oxidative oligosaccharides and the content of undesired interfering substances (such as e.g. chlorophyll, carotenoids and other, in particular lipophilic, components).

The precise conditions of the extraction are in each case determined in preliminary experiments using analytics for anti-oxidative oligosaccharides which shall be contained in the extracts in a minimum amount, as mentioned above.

Following extraction a centrifugation is performed in a flow centrifuge (“milk centrifuge”) to separate the insoluble components. The separated insoluble components (rape) are discarded. The supernatant (or overflow) is chilled to 4 degrees Celsius but at least to 40 degrees Celsius. After a storage period of up to one month the precipitating lipophilic components (in particular chlorophyll) are removed by means of filtration.

Afterwards, the filtrate is concentrated at 50 to 90 degrees Celsius in vacuo to about one tenth to one quarter of the original volume. The concentrated solution is again refrigerated down to 4 degrees Celsius and at least to 40 degrees Celsius. Following this final clarification the precipitating or flocculating suspended matter is removed after a storage period of up to one month by means of filtration.

Subsequently, to the filtrate is added an inert material, e.g. maltodextrine or aerosil, to obtain a desired concentration of anti-oxidative oligosaccharides in the final product. The final drying step is performed in a vacuum concentrator or on a drying conveyor belt at temperatures between 40 and 90 degrees Celsius. Afterwards, the dried material is ground in a mill to the desired grain size. The powder is packaged under vacuum. The final product which is suitable for the preparation of beverages dissolves in water without remainder (especially, no coloured particles (black “dots”) segregate). The final product contains a minimum amount of anti-oxidative oligosaccharides of about 10% and fulfils the minimum criteria for microbiological purity according to food regulations and pharmacopoeias. It also remains under and thus keeps the upper limits of heavy metals, herbicides, and pesticides regulated by law.

### Example 2

### Anti-inflammatory Efficacy of the Extract of Aloysia triphylla in the HET-CAM Test

For this purpose, 10 μl of a solution of 50 mg lyophilisate (of the composition obtained in Example 1 above) in 1 ml of agarose solution (containing 5 mg lauryl sulfate/ml) were applied to the CAM as the test pellet. The angiogenesis caused by the inflammation appeared less or was even completely suppressed in the area of the test pellet in comparison to the control experiment. This experimental layout is an acknowledged model for in vivo examination of the inhibition of angiogenesis and proves the completely unexpected novel pharmacological effects of the above-mentioned lyophilisates. In this respect, see also FIG. 4.

The HET-CAM test is one of a number of model tests to examine substances with respect to their inhibition of pathogenic angiogenesis for possible therapeutic applications. The advantage of the HET-CAM test is that it belongs to those in vivo experiments enabling a more definite prediction regarding clinical relevance than in vitro methods. This in vivo test contains the complex system of angiogenesis with all its cellular functions and mediators enabling a relatively certain prediction with respect to inhibitory action on angiogenesis. The test is appreciated as a screening procedure for the detection of substances having angiogenesis-inhibiting properties (Svahn, C. M., M. Weber, C. Mattsson, K. Neiger, M. Palm Carbohydr. Polym. 18, 9-16 (1992); Hahnenberger R., A. M. Jakobsen, A. Ansari, T. Wehler, C. M. Svahn, U. Lindahl, Glycobiology 3, 567-573 (1993); and Galliardi, A., H. Hadd, D. C. Collins, Cancer Research 52, 5073-5075 (1992)).

### Example 3

### Confirmation of the Results Obtained in the HET-CAM Test in an Animal Experiment

The results of the HET-CAM assay could be furthermore confirmed in an animal model of chronic inflammation. The effect of the extract was examined in a mouse model of dextrane sulfate-induced acute colitis.

For this purpose, an acute colitis is induced in a total of 10 mice by means of dextrane sulfate. Due to this intestinal inflammation the mice loose weight. Terminal parameters are determination of the mouse weight, the intestinal length and the reduction of known interleukins causing inflammation.

Following the administration of 600 μg of water-soluble Aloysia extract daily (over 10 days) the final weight of 5 mice after 10 days of treatment was significantly increased (p=0.05) as compared to the control group (5 mice). The results of the treatment are presented in Table 1. The values listed are mean values with the maximal ranges of variation.

TABLE 1

| | Starting weight in grams | Final weight in grams |
|---|---|---|
| Treatment | (day 0) | (day 10) |
| 600 μg extract of the | 21.2 (+/−0.8) | 19.2 (+/−1.2) |
| invention (see above) | | |
| PBS (control) | 20.8 (+/−0.5) | 15.4 (+/−1.4) |

The values of IFN and IL-10 were significantly reduced after stimulation in MLN cells obtained from lymph knots.

These results of the mouse experiment confirm the successful use of the composition according to the invention in chronic inflammation, in particular of the intestinal tract.

### Example 4

### Preparation of Foodstuffs Containing the Extract According to the Invention

Starting product: water-soluble fraction of an alcoholic extract from Aloysia triphylla (obtained as in Example 1 above), optimised to a content in anti-oxidative oligosaccharides of about 10%).

Table 2 shows the mixing ratios of the extract in each of the dairy products. The results listed in the Table were obtained.

TABLE 2

| | yogurt | fresh cheese | milk shake |
|---|---|---|---|
| | (strawberry) | (herb-flavoured) | (basic mix) |
| | ELS 04-06/2003 | ELS 04-06/2003 | ELS 10/2003 |
| dosage | a) 1000 mg/150 g | a) 3000 mg/200 g | a) 750 mg/1 l |
| | b) 500 mg/150 g | b) 1000 mg/200 g | b) 500 mg/1 l |
| | c) 250 mg/150 g | c) 500 mg/200 g | c) 250 mg/1 l |
| taste | d) bitter | d) none | d) none |
| | e) slightly bitter | e) none | e) none |
| | f) slightly bitter | f) none | f) none |
| colour | g) slightly brownish | g) slightly brownish | g) brownish |
| | h) neutral | h) neutral | h) slightly brownish |
| | i) neutral | i) neutral | i) neutral |
| odour | none | none | none |
| miscibility | fully miscible | fully miscible | fully miscible |
| solubility | Fully soluble | fully soluble | fully soluble |
| stability | 90 days in aqueous | 90 days | 90 days |
| | acidic solution (pH 4) | | |
| storage stability | at least 28 days | at least 28 days | at least 28 days |
| in the product | | | |
| microbiology | o.k. | o.k. | o.k. |
| scaling up | 1000 kg each of | 1000 kg of herb- | 3000 l of |
| | plum-muesli yogurt | flavoured | chocolate milk |
| | apple-muesli yogurt | fresh cheese | shake |
| recommended | in aromatised or | in aromatised or | in tastes such as |
| use | muesli yogurts; | herb-flavoured | chocolate, mocca, nut etc. |
| | in the fruit preparation | fresh cheeses; | |
| | | in the fruit | |
| | | preparation | |

For extracts of Aloysia triphylla (Lemon Verbena) no recent pharmacological studies are available. The known studies usually have investigated the essential oil. The leaves of the small shrubs also called Verbenae contain large amounts of this essential oil the odour of which has fostered the wide distribution as an ornamental plant.

The drug or the extracts, respectively, are traditionally accepted by the approving authorities in France for the symptomatic treatment of digestive troubles on the one hand and of tenseness and sleeping disorders on the other hand.

Up to now, Aloysia triphylla finds use in the food industry in the form of herbal teas. In the cosmetic industry, the essential oils obtained from the plant are used as fragrances. The use of aqueous or ethanolic-aqueous (hydrophilic) extracts is not known.

It has now been surprisingly found that extracts of Aloysia triphylla, in particular lyophilisates thereof, have unexpected novel pharmacological effects which are neither described in the prior art nor suggested by the known ingredients and prior indications of the drug.

### SUMMARY

Therefore, it is an object of the present invention to provide a novel and broadly useful extract of Aloysia triphylla.

This object has been achieved by the subject matter of the independent claims. Preferred embodiments are set forth in the dependent claims.

In the following, the present invention will be illustrated by FIGS. and Examples. It should be understood, however, that it is not limited thereto, its scope is rather defined by the claims.

What is claimed is:

1. A method for the preparation of an extract of Aloysia triphylla comprising the following steps:
a) providing of dried herb of Aloysia triphylla;
b) extracting the herb with an aqueous-ethanolic extractant;
c) centrifugation and separating the insoluble residue from the extract;
d) filtration of the extract to separate lipophilic components, particularly chlorophyll;
e) concentrating, clarifying and drying said extract.

2. An extract which is obtainable by the method according to claim 1.

3. The extract according to claim 2 wherein the extract is essentially completely soluble in water.

4. The extract according to claim 2 or 3 wherein the extractant employed for extraction contains water and/or ethanol.

5. The extract according to claims 2-4 wherein the extractant contains water in a range of 100% V/V to 30%V/V.

6. The extract according to one or more of the preceding claims wherein the extract is a fluid extract or a specific extract.

7. The extract according to one or more of the preceding claims which is present in a lyophilised form.

8. A foodstuff, nutraceutical or cosmetic containing an extract of Aloysia triphylla according to one or more of claims 2-7.

9. A pharmaceutical composition containing an extract of Aloysia triphylla according to one or more of claims 2-7 and one or more pharmaceutical adjuvants/carriers.

10. The pharmaceutical composition according to claim 9 which is designed for administration by injection, systemic and/or topic administration.

11. The use of an extract according to one or more of claims 1-7 for chemoprevention and treatment of rheumatoid arthritis, chronic intestinal diseases, and colitis.

12. The use according to one or more of claims 10-11 wherein the dosage of the fluid extract is between 20 mg and 2 g per day based on dry substance.
